(19) United States
(12) Plant Patent Application Publication
(10) Pub. No.: US 2004/0250324 P1
(43) Pub. Date: Dec. 9, 2004

(54) Miniature rose plant 'PACfirst'

(76) Inventor: Dan Jauchen, Santa Barbara, CA (US)

Correspondence Address: Dan Jauchen, 905 South Patterson Avenue, P.O. Box 6354, Santa Barbara, CA, 93160, US

(21) Appl. No.: 10/454,455

(22) Filed: Jun. 5, 2003

(51) Int. Cl. A01H 5/00

(52) U.S. Cl. PLT/122

(57) ABSTRACT

A new miniature rose plant which has abundant, orange colored flowers and attractive foliage. The variety successfully propagates from softwood cuttings and is suitable for year round production in commercial glass houses as a flowering pot plant. This new and distinct variety has shown to be uniform and stable in the resulting generations from asexual propagation.

### LATIN NAME OF GENUS AND SPECIES

Rosa hybrid ‘PACfirst’.

### VARIETY DENOMINATION

The new variety is named ‘PACfirst’.

### BACKGROUND OF THE INVENTION

The present invention constitutes a new and distinct variety of miniature rose plant, which was developed by artificially pollinating an unnamed seedling (not patent in the US) with an unnamed seedling (not patent in the US). The two parents were crossed in the summer of 1999 and the resulting seed was sown in December, 1999 in a controlled glasshouse environment. Out of these seedlings one seedling was selected, as the new variety and named ‘PACfirst’. The new rose may be distinguished from its seed parent, an unnamed seedling, by the following combination of characteristics:

1. The unnamed seedling has a breeding background in unnamed seedlings.

2. ‘PACfirst’ has medium double flowers, while the unnamed seedling has big double flowers.

3. ‘PACfirst’ has orange colored petals, while the unnamed seedling has dark yellow petals.

The new variety may distinguished from its pollen parent, an unnamed seedling created by the same inventor, by the following combination of characteristics:

1. The unnamed seedling has a breeding background in unnamed seedlings.

2. ‘PACfirst’ has smaller flowers and foliage as compared to the unnamed seedling.

3. ‘PACfirst’ has orange colored petals, while the unnamed seedling has dark red petals.

### BRIEF SUMMARY OF THE INVENTION

Initial asexual reproduction of ‘PACfirst’ by cuttings was first done in Santa Barbara, Calif., USA. The reproduction was conducted in controlled greenhouse environments.

Have here proven to be stabile by propagation with cuttings in several generations. ‘PACfirst’ is a low and compact miniature rose with medium vigor. The shelflife has been tested to be medium.

The objective of the hybridization of this rose variety for commercial greenhouse culture was to create a new and distinct variety with:

1. Uniform and abundant flowers with good keepability;

2. Attractive long lasting foliage and compact growth,

3. Year round flowering under glasshouse conditions;

4. Suitability for production from softwood cuttings in pots;

5. Durable flowers and foliage which make the variety suitable for distribution in the floral industry.

This combination of qualities was not present in previously available commercial cultivars of this type and distinguish ‘PACfirst’ from all other varieties of which we are aware.

The seeds from hybridization were planted in a controlled environment and evaluations were conducted on the resulting plants. ‘PACfirst’ was selected by, Dan Jauchen, in his development program in Santa Barbara, Calif., USA.

### BRIEF DESCRIPTION OF THE DRAWINGS

The accompanying color illustrations show as true as is reasonably to obtain in color photographs of this type, the typical characteristics of the buds, flowers, leaves, stems of ‘PACfirst’. Specifically illustrated in:

Photo Sheet #1:

FIG. 1. Young shoot.

FIG. 2. Bud before opening the sepals.

FIG. 3. Bud at the opening the sepals.

FIG. 4. Bud at the opening the petals.

FIG. 5. Flower during course of opening.

FIG. 6. Open flower—plan view—obverse.

FIG. 7. Open flower—plan view—reverse.

FIG. 8. Fully open flower—plan view—obverse.

FIG. 9. Fully open flower—plan view—reverse.

Photo Sheet #2:

FIG. 10. Receptacle showing stamens and pistils.

FIG. 11. Receptacle showing pistils (stamens removed).

FIG. 12. Flower petals, detached—outer surface.

FIG. 13. Flower petals, detached—inner surface.

FIG. 14. Bare stem exhibiting thorns and flower attachment.

FIG. 15. Three leaflets upper side.

FIG. 16. Three leaflets reverse side.

FIG. 17. Five leaflets upper side.

FIG. 18. Five leaflets reverse side.

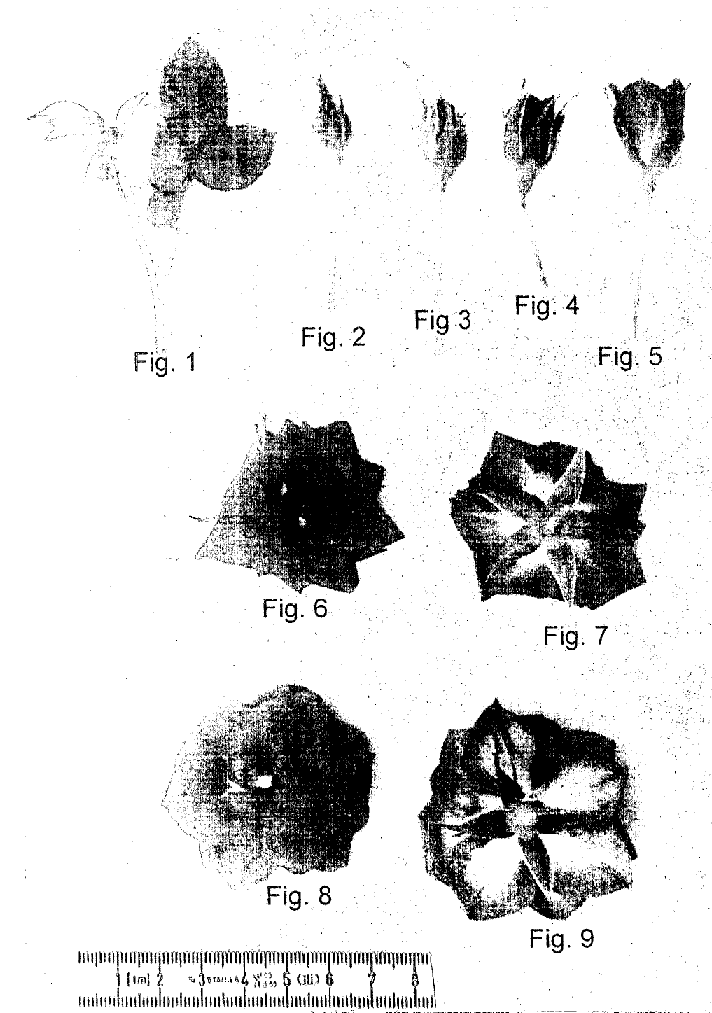

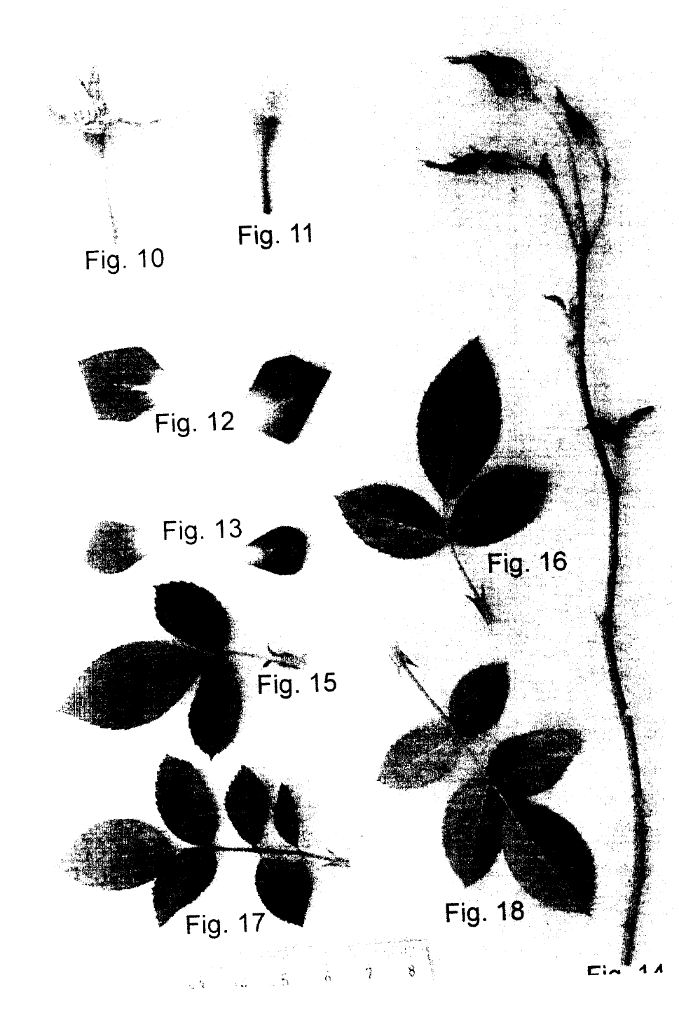

### DETAILED BOTANICAL DESCRIPTION OF THE VARIETY

The following is a detailed description of the Miniature Rose: Rosa hybrid ‘PACfirst’.

The following observations, measurements, values and comparisons describe plants grown in glass houses in Santa Barbara, Calif., USA.

The age of the observed plants where 11 to 13 weeks after propagation by cuttings, and produced as flowering pot plants in container of 10.5 centimeter in diameter.

Color references are made using The Royal Horticultural Society (London, England) Colour Chart, 1995, except where common terms of color are used. For a comparison, the nearest existing rose variety is ‘POUfiry’, a rose variety described and illustrated in U.S. Plant Pat. No. PP12,484.

Chart 1 details several physical characteristics of ‘PACfirst’ and ‘POUfiry’.

| Chart 1: | ‘PACfirst’ | ‘POUfiry’ |
|---|---|---|
| Petal color, Upper | Orange-Red Group | Red Group |
| surface | 34 A | 40B-C |
| Petal color, Reverse | Orange-Red Group | Red Group |
| surface | 34A | 41C |
| Petal count | 25-30 | 20-25 |

Parents.—Unnamed seedling. Times. Unnamed seedling.

Classification:

Botanical.—Rosa hybrid.

Commercial.—Miniature.

Plant:

Plant growth.—Moderately vigorous. Grows compact upright to bushy. When grown as 10 cm pot plant, the average height of the plant itself is 18 to 20 cm, and average with is 20 cm. When grown as a 15 cm pot plant, the average height of the plant itself is 22 to 27 cm, and average with is 30 cm. Production time is generally 11 to 13 weeks depending on average temperature, light level, and cultural practices.

Stem:

Color.—Young wood: Yellow-Green Group 144B. Older wood: Yellow-Green Group 144A.

Thorns.—Incidence: Medium number of thorns Size: 4-5 mm Color: Greyed-Yellow Group 161D Shape: Deep concave.

Surface.—Young wood: Smooth Older wood: Smooth.

Stem diameter.—3-4 mm.

Internode length.—30-35 mm.

Numbers of internodes.—6-8.

Plant foliage: Leaves arranged alternately, compound with three to seven leaflets per leaf, generally symmetrical, abundant, and flat in aspect. Stipules at petiole base.

Quantity of leaves.—6 to 8 per lateral branch.

Leaf size.—Medium 85-90 mm(l). times. 50-55 mm(w).

Petioles.—Color: Yellow-Green Group 144B, with intonations of Greyed-Purple Group 183C Margins: with stipitate glands Length: 10-15 mm Diameter: about 0.5-1 mm.

Stipules.—Size: 6-7 mm Surface: Smooth Color: Yellow-Green Group 145B, with intonations of Yellow-Green Group 144B Margins: with stipitate glands.

Rachis.—Color: Yellow-Green Group 144A, with intonations of Greyed-Purple Group 183C Margins: Margins with stipitate glands. Length: 20 to 40 mm.

Leaflets.—Edge: Serrated Serration: Single Shape: Ovate with acute apex and obtuse base Texture: Smooth Appearance: Glossy Size: length: 20 to 30 mm. With: 12 to 30 mm Color: Young foliage: Upper surface: Green Group 137C Lower surface: Yellow-Green Group 148C, With intonations of Greyed-Purple 183C Color: Mature foliage: Upper surface: Green Group 137B Lower surface: Yellow-Green Group 147B.

Inflorescense:

Blooming habit.—Recurrent.

Number of flowers.—Generally 1 to 4 bud per flowering stem.

Penduncle.—Color: Yellow-Green Group 144C, Texture: Smooth, with pubescence and prickles Length: 30-40 mm Strength: Upright.

Receptacle.—Surface: Smooth, glabrous. Shape: Funnel-shaped Size: h: 5-6 mm w: 4-5 mm Color: Yellow-Green Group 144C.

Sepals.—Quantity: 5 Shape: Narrowly Ovate w. acute tip Texture: Leathery Margin: Foliaceous appendages on 3 of the five sepals Appearance: Dull Color: Upper surface: Green Group 138B Reverse surface: Greyed-Green Group 143B.

Buds.—Size: 20-25 mm (h) 15-20 mm (w)upon opening. Shape: Cupped Color: at ¼ opening, Red Group 52A.

Flower:

Duration.—As a pot plant, flowers last form 12 to 18 days. Fragrance. None. Size: 50-55 mm in diameter. Form: Shape of flower when viewed from the side. Up on opening: Cupped Open flower: Flat Color: Petals, upon opening. Upper surface: Orange-Red Group 34A, Reverse surface: Orange-Red Group 34A, Petals after opening: Upper surface: Orange-Red Group 34A. Reverse surface: Orange-Red Group 34A. Basal Petals spots: Size: 2-3 mm Color: White Group 155D General tonality: On Open flower: Third day: Orange-Red Group 34A Afterwards: Orange-Red Group 34A-B.

Petals:

Petal reflex.—Outermost petals reflex backwards at opening. Fully open all petals reflex backwards.

Texture.—Smooth.

Petal edge.—Uniform.

Petal count.—Approximately 25-30 on the average per flower.

Petal size.—Length 20 mm With: 20 mm.

Shape.—Outer petals: Round-ovate Inner petals: Ovate.

Reproductive organs:

Stamen number.—Approximately 55-70 on average per flower.

Pollen.—Color: Yellow-Orange Group 22A, Abundance: Average.

Anthers. Size: 1-2 mm Color: Yellow Group 2D, Shape: Oblong.

Filaments.—Size: 4-5 mm Color: Red-Purple Group 58B.

Pistils number.—Approximately 25-35 on average per flower.

Stigmas.—Location: Superior in location to anthers Color: Yellow-Green Group 145C.

Styles.—Color: Yellow-Green Group 145C. Length: 3 to 4 mm.

Development:

Vegetation.—Dense.

Blooming.—Abundant.

Aptitude to bear fruit.—Poor.

Resistance to diseases.—Above average resistance to mildew and Botrytis under normal growing conditions in Santa Barbara, Calif., USA. Hips/seeds has not been observed due to that the plant has never been grown to the stage of seed development, due to the fact, that the variety is developed for use as a flowering pot plant only.

Winter hardiness & drought/heat tolerance.—Due to the fact, that this variety is a potted flowering plant, developed for one time use only, the plant is not tested for winter hardiness or drought/heat tolerance.

1. A new and distinct variety of rose plant of the miniature class, substantially as herein illustrated and described as a distinct and novel rose variety due to its abundant orange flowers, attractive long lasting foliage, vigorous and compact growth, year round flowering under glasshouse conditions, suitability for production from softwood cuttings in pots, and durable flowers and foliage which make the variety suitable for distribution in the floral industry.
